(19) United States
(12) Patent Application Publication
(10) Pub. No.: US 2008/0318241 A1
(43) Pub. Date: Dec. 25, 2008

(54) Methods and Systems for Detecting Antiangiogenesis

(75) Inventors: Long Hoang Dang, Ann Arbor, MI (US); Ngoc-Duyen Thi Dang, Ann Arbor, MI (US); Nam Hoang Dang, Las Vegas, NV (US)

Correspondence Address: Casimir Jones, S.C., 440 Science Drive, Suite 203, Madison, WI, 53711, US

(73) Assignees: THE REGENTS OF THE UNIVERSITY OF MICHIGAN, Ann Arbor, MI (US); NEVADA CANCER INSTITUTE, Las Vegas, NV (US)

(21) Appl. No.: 12/140,778

(22) Filed: Jun. 17, 2008

Related U.S. Application Data
(60) Provisional application No. 60/936,039, filed on Jun. 18, 2007.

(51) Int. Cl. C12Q 1/68; C12Q 1/37

(52) U.S. Cl. 435/6; 435/24

(57) ABSTRACT

The present invention provides methods and systems for the detection of tumor vessel response to antiangiogenic therapies. The present invention also provides compositions and methods for therapeutic and research applications. In particular, the present invention provides systems and methods that employ CD26, HIF-1, and HIF-1 pathway components as biomarkers for monitoring antiangiogenic therapies and as therapeutic targets.

This application claims priority to provisional patent application Ser. No. 60/936,039, filed Jun. 18, 2007, which is herein incorporated by reference in its entirety.

This invention was made with government support under Grant No. K22CA111897 awarded by the National Institute of Health. The government has certain rights in the invention.

### FIELD OF THE INVENTION

The present invention provides methods and systems for the detection of tumor vessel response to antiangiogenic therapies. The present invention also provides compositions and methods for therapeutic and research applications. In particular, the present invention provides systems and methods that employ CD26, HIF-1, and HIF-1 pathway components as biomarkers for monitoring antiangiogenic therapies and as therapeutic targets.

### BACKGROUND OF THE INVENTION

Treatments with the goal of inhibiting tumor blood vessels have recently been shown in phase III clinical trials to improve survival in patients with advanced stage cancers of various subtypes. Two seemingly opposing views have been raised to explain the mechanism of inhibition of tumor progression with antiangiogenic therapy. First, antiangiogenic therapy is thought to lead to tumor vessel normalization, decreased interstitial fluid pressure, and hence improved delivery of chemotherapy. Second, antiangiogenic therapy is thought to lead to inadequate tumor vessels and thus starving tumor cells of oxygen and nutrients. Both of these viewpoints have been supported by preclinical and clinical data, showing either normalized, or inadequate vessels, in response to antiangiogenic therapy (Jain, 2005, Science 307:58-62; Folkman, 2002, Semin. Oncol. 29:15-8). There are currently no reliable predictors of antiangiogenic response (Hurwitz et al., 2004; Yang et al., 2003). A better understanding of the determinants, and consequences, of these two types of vessel responses would have significant clinical implications.

Vascular endothelial growth factor A (VEGFA or VEGF) is the most ubiquitous activator of tumor angiogenesis (Brown et al., 1993, Cancer Res. 53:4727-35; Carmeliet et al., 1996, Nature 380:435-9; Ferrara et al., 1996, Nature 380:439-42; Grunstein et al., 1999, Cancer Res. 59:1592-8; Gale et al., 2002, Cold Spring Harb. Symp. Quant. Biol. 67:267-73; Fukumura et al., 1998, Cell 94:15-25; Duda et al., 2004, Cancer Res. 64:5920-4). In fact, the incorporation of agents targeting VEGF, or VEGF receptors, has been a major milestone in the treatment of cancer. In phase III clinical trials, addition of the humanized anti-VEGF monoclonal antibody, bevacizumab, to standard chemotherapy, led to increased tumor response and duration of response; in patients with advanced colorectal, breast, and lung cancers (Fernando et al., 2003, Semin. Oncol. 30:39-50; Hurwitz et al., 2004, N. Engl. J. Med. 350:2335-42; Yang et al., 2003, N. Eng. J. Med. 349:427-34; Burkowski, 2004, Curr. Oncol. Rep. 6:85-6; Willett et al., 2004, Nat. Med. 10:145-7; Ferrara et al., 2003, Nat. Med. 9:669-76; McCarty et al., 2003, Trends Mol. Med. 9:53-8). In Phase III clinical trials, treatment with the multitargeted tyrosine kinase inhibitors sunitinib and sorafenib, which block VEGFR2 along with other kinases, significantly improved time-to-progression; in patients with GIST and renal cell cancer.

What are needed are methods and systems for determining the antiangiogenic response of therapeutic antiangiogenic therapies, such as identifying markers correlated with antiangiogeneic therapeutic response. What are also needed are improved therapeutic compositions and methods that work via control of angiogenesis or other mechanisms. Such determinations would be useful in clinical diagnostics and therapeutics, drug discovery, and research efforts studying, for example, cancer and other angiogenic related diseases.

### SUMMARY OF THE INVENTION

The present invention provides methods and systems for the detection of tumor vessel response to antiangiogenic therapies. The present invention also provides compositions and methods for therapeutic and research applications. In particular, the present invention provides systems and methods that employ CD26, HIF-1, and HIF-1 pathway components as biomarkers for monitoring antiangiogenic therapies. Further, methods and systems of the present invention provide for the use of HIF-1 and HIF-1 pathway component inhibition in cancer therapies.

Antiangiogenic therapy has been an attractive approach to treat cancer and angiogenic related diseases for multiple reasons. For example, neovascular growth is necessary for cancer cells to grow into a clinically significant mass. Targeting vasculature would lead to a significant bystander effect, as each blood vessel supports many cancer cells. As endothelial cells are located within the vasculature, drug delivery would not have to overcome the same barriers as standard chemotherapeutics such as distance, high intratumoral pressure, and hypoxia. As well, endothelial cells are genetically stable and less likely to give rise to therapy-resistant clones.

In some embodiments, the present invention provides methods for determining a response to antiangiogenic treatment comprising: providing a sample from a subject undergoing antiangiogenic treatment, assaying the sample for CD26 expression and determining a response to antiangiogenic treatment based on the expression of CD26 in the sample (e.g., relative to that of a control, a prior measurement, or a threshold value). In some embodiments, the sample is serum and/or a tissue sample, which is further either a biopsy or a tissue lysate, although the present invention is not limited by the nature of the sample used. In some embodiments, the subject is a mammal, preferably a human. In some embodiments, the assay used in determining CD26 expression is a protein and/or a nucleic acid based assay. In some embodiments, an increased expression in CD26 is seen indicative of normalization of abnormal tumor vessels associated with one type of antiangiogenic response. In some embodiments, a decrease in expression in CD26 is seen indicative of tumor vessel regression and/or tumor tissue hypoxia that is associated with another type of antiangiogenic response.

In some embodiments, the present invention provides a method for determining the prognosis of an antiangiogenic treatment comprising providing a sample from a patient undergoing antiangiogenic treatment, assaying for CD26 expression, and determining a prognosis of said treatment based on CD26 expression.

In some embodiments, the methods employ monitor HIF-1 expression or activity or the expression or activity of a HIF-1 pathway member.

In some embodiments, the present invention provides a method for determining inhibition of HIF-1 by a compound comprising: providing a sample in the presence or absence of said compound, assaying for expression or activity of HIF-1 or a HIF-1 pathway member (e.g., CD26), and determining direct or indirect inhibition of HIF-1 by said compound based on the expression or activity level of the HIF-1 pathway member.

The present invention further provides methods of treating a patient having a cancer that comprise administering to the patient, a pharmaceutical formulation comprising an agent (e.g., antibody, small molecule drugs, antisense oligonucleotide, siRNA, peptide, etc.) that inhibits or activates a HIF-1 pathway member (e.g., other than CD26) to inhibit cell growth.

The present invention further provides systems, compositions, and kit comprising components useful, necessary, or sufficient for carryout out the above methods.

### DEFINITIONS

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “providing a prognosis” in the present application refers to providing information regarding the impact of an antiangiogenic treatment or therapy on the presence, degree, or type of cancer (e.g., normoxic or hypoxic) (e.g., as determined by the methods of the present invention) on a subject's future health. In some instances, the prognosis allows a clinician to augment treatment for a patient to a treatment regimen that is more beneficial in inhibiting or decreasing angiogenic tumors in the patient.

As used herein, the term “biopsy tissue” refers to a sample of tissue (e.g., tumor tissue) that is removed from a subject for the purpose of determining, for example, if the sample contains cancerous tissue or for use in in vitro analysis using methods and systems of the present invention.

As used herein, the term “non-human animals” refers to all non-human animals including, but are not limited to, vertebrates such as rodents, non-human primates, ovines, bovines, ruminants, lagomorphs, porcines, caprines, equines, canines, felines, aves, etc.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein the terms “test compound” “therapeutic test compound” and treatment test compound” refer to any chemical entity, pharmaceutical, drug, and the like that is a candidate for use to treat or prevent a disease, illness, sickness, or disorder of bodily function, such as use as an antiangiogenic compound or an inhibitor of HIF-1. Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, and tissues. Biological samples include blood products, such as plasma, serum and the like and tissue samples, such as biopsy samples and the like. Such examples are not however to be construed as limiting the sample types applicable to the present invention.

### DESCRIPTION OF THE FIGURES

FIG. 1 demonstrates the disruption of VEGF in cancer cell lines. A, Disruption of VEGF. The endogenous VEGF locus, AAV knockout construct, and resulting targeted locus are shown. Numbered boxes represent exons. Gray boxes represent targeted exon 2. ITR=inverted terminal repeats; HA=homology arm; P=SV40 promoter; Neo=neomycin-resistance gene; pA=polyA tail; striped triangles=loxP sites; P1/P2=primers for locus-specific PCR. B, Locus-specific PCR to confirm homologous integration of the targeting vector. Lane 1: VEGF+/+ cells: only the native loci (˜240 bp) are amplified. Lane 2: VEGF+/−cells: the approximately 2036 bp product is the locus with targeting vector inserted, and the approximately 240 bp product is the native locus on the 2nd allele. Lane 3: VEGF+/−cells: the approximately 360 bp product is the disrupted locus, with Neo removed by Cre recombinase. Lane 4: VEGF−/− cells: the approximately 2036 bp product is the 2nd locus with the targeting vector inserted; the approximately 360 bp product is the disrupted locus on the 1st allele. C, ELISA for human VEGF.

FIG. 2 shows exemplary tumor growth, microvessel density, and blood flow in VEGF+/+ and VEGF−/− xenografts. A, Examples of xenografts. Bar=1.0 cm. B, Time to tumor progression to 0.2 cm³. Xenograft volumes were plotted as a function of time. The number of days for tumors to reach 0.2 cm³ was calculated. N=10 for each symbol, *p<0.01 comparing VEGF−/− to parental xenografts.

FIG. 3 shows the analyses of intratumoral hypoxia, expression of HIF-1α and HIF-1 target genes in VEGF+/+ and VEGF−/− xenografts. A, Immunohistochemistry for pimonidazole adducts, an indicator of hypoxia, in tumor xenografts. B, Western blot for HIF-1α expression in tumor xenografts. C, Expression of HIF-1α target genes in xenografts. ALDOA, GLUT1, LDHA, PFKL, and CD26 (DPP4), relative to β-actin, were measured by real time RT-PCR and values graphed relative to parental HCT116 xenografts values. N=3 for each bar. *=p<0.01 comparing the VEGF−/− xenografts with parental VEGF+/+ xenografts. **=p<0.01 comparing VEGF−/− HIF-1α−/− to VEGF−/− xenografts.

FIG. 4 demonstrates the expression of HIF-1α target genes in LS174T and MKN45 cell lines. ALDOA, GLUT1, LDHA, PFKL and CD26 (DPP4), relative to β-actin were measured by real-time RT-PCR. N=3 for each bar. *=p<0.01 comparing hypoxic (1% oxygen culture for 16 hours) to normoxic (21% oxygen culture) conditions.

FIG. 5 shows exemplary known HIF-1 transcriptional targets.

### DETAILED DESCRIPTION OF THE INVENTION

Vascular endothelial growth factor A (VEGFA or VEGF) is the most ubiquitous activator of tumor angiogenesis. From animal studies of blood vessels development in embryos, tumors, and diabetic retinopathy, it is thought that newly formed vessels are more dependent than established vessels on VEGF for survival.

One difference between newly formed remodeling vessels, as compared to ones seen in tumors and mature vessels in body organs, is the extent of coverage by mural cells. Mature blood vessels contain endothelial tubes that are surrounded by mural cells; whereas remodeling tumor vessels contain a significant fraction of vessels that are devoid of mural cells.

Based on ultrastructural studies, vascular smooth muscle cells have been identified as the mural cells of arteries, arterioles, and veins; while pericytes are the mural cells of capillaries and venules. An important role of mural cells in maintaining vascular integrity has been demonstrated in a number of gene knockout studies in mice. Blood vessels of mouse embryos lacking platelet-derived growth factor-BB (PDGF-β) or its receptor PDGFR-β, angiopoietin-1 (Ang-1) or its receptor Tie-2, endoglin, and tissue factor (TF) are abnormally large, leaky and associated with deficient pericytes or vascular smooth muscle cells coverage. These mice died in utero as a result of vascular defects.

Based on these observations, it is contemplated that mural cells mediate stabilization of endothelial tubes as blood vessels mature. Mural cells support endothelial cells both by producing paracrine signals through the secretion of VEGF and angiopoietin-1 (Ang-1) and by providing stabilizing interactions through the expression of adhesion receptors integrin α4β1 and N-cadherin. In studies of vessels regression either in tumors upon growth factor withdrawal or in diseases of the retina, vessels regression was primarily due to a selective loss of immature, pericyte-negative vessels and an increase in the fraction of more mature, pericyte-positive vessels (Abramovitch et al., 1999, Cancer Res. 59:5012-6; Gee et al., 2003, Am. J. Path. 162:183-93; Benjamin et al., 1999, J. Clin. Inv. 103:159-65). The selective loss of endothelial cells not protected by pericytes is due to their dependence on VEGF for survival. When both endothelial cells and pericytes are targeted by inhibitors, vessel regression was further induced (Takagi et al., 2003, Inv. Opthal. Vis. Sci. 44:393-402).

Although the importance of pericytes in stabilizing endothelial cells is becoming clearer, it is not well understood what pericyte subpopulation best carries out this function. Pericytes are characterized not only by their distinctive shape and location surrounding endothelial tubes, but also by their expression of various markers including α-smooth muscle actin (SMA), desmin, PDGFR-β, calponin, caldesmon, tropomyosin, and high-molecular weight melanoma-associated antigen (NG2). Pericytes in different types of blood vessels, organs, and pathological conditions vary in the relative expression of these markers. In the retina, pericyte expression of more differentiated markers correlated with vessel stability. Most studies in cancer have only looked at one marker —NG2, SMA, or desmin- and have equated lack of expression of one particular marker to the absence of pericytes.

In preclinical models, blocking VEGF signaling either by inhibitors of VEGF or VEGF receptors resulted in impaired tumor angiogenesis and growth. In phase III clinical trials, the addition of humanized anti-VEGF monoclonal antibody, bevacizumab, over standard therapy alone led to improvement in objective tumor response and overall improved duration of response, which were statistically significant for patients with advance colorectal, breast, and lung cancers. Phase III clinical trials using sunitinib or sorafenib, multitargeted tyrosine kinase inhibitors that block VEGFR2 along with other kinases, showed significant improvement in time-to-progression in patients with GIST and renal cell cancer, respectively.

While these treatment approaches targeting VEGF or VEGF receptors represent significant milestones in the treatment of cancer, the mechanism of antiangiogenic efficacy has not been clearly elucidated. Furthermore, there are no reliable predictors of response. Two seemingly conflicting theories have been proposed to explain the antiangiogenic mechanism. The first theory proposes that antiangiogenic therapy leads to normalization of abnormal tumor blood vessels, which then drops interstitial pressure within tumors and facilitates intratumoral delivery of chemotherapy for improved efficacy. In the second theory, it is thought that effective antiangiogenic therapy produces inadequate blood vessels, depriving the tumor mass of oxygen and nutrients, and thus inhibiting tumor growth. Both viewpoints are supported by preclinical data showing vessels response in some situations consistent with normalized and others with inadequate blood vessels.

In developing embodiments of the present invention, it was contemplated that heterogeneity of the tumor vasculature in both experimental tumors and primary patient tumors is responsible for the different types of vessel response. In evaluating a panel of 12 human cancer xenografts grown in nude mice, significant heterogeneity in microvessel density, endothelial tube length, pericyte subpopulations, and extent of interactions between endothelial tubes and specific pericyte subpopulations was found. Comparing immunoreactivity for the two pericyte markers, NG2 and SMA, it was found that there are two distinct pericytes subpopulations that are distinguished by these two markers: NG2+SMA− and NG2+SMA+. Amongst the xenografts, there is heterogeneity in both quantity and relative abundance of these two types of pericytes. Next, it was investigated whether there was difference in the ability of NG2+SMA− and NG2+SMA+ pericytes in protecting endothelial cells from VEGF inhibition. To control for variability in perfusion by antibody or small-molecule inhibitors secondary to the heterogeneity in vasculature amongst the xenografts, somatic knockout was used in the model as described herein to shut off VEGF secretion from the cancer cells. It was found that NG2+SMA+, but not NG2+SMA−, pericytes protect endothelial cells from VEGF inhibition. In xenografts with high density of NG2+SMA+ pericytes (LS174T and MKN45), there was better preservation of the tumor vasculature in the absence of tumor cell-derived VEGF. In xenografts with low density of NG2+SMA+ pericytes (HCT116 and RKO), there was only short residual endothelial stumps with poor perfusion.

As VEGF inhibition leads to distinct vessels response phenotype consistent with either normalized or inadequate blood vessels, it was further contemplated that tumor oxygenation is differentially affected. It was found that tumors with normalized blood vessels showed no noticeable change in tumor oxygenation as measured by an exogenous marker, pimonidazole, or endogenous markers reflective of cellular oxygenation, expression of HIF-1α and HIF-1 target genes. Contrarily, tumors with inadequate blood vessels showed increased intratumoral hypoxia, stabilization of HIF-1α, and induction of HIF-1 target genes.

In developing embodiments of the present invention, the heterogeneity of the tumor vasculature and coverage by pericytes was investigated. Twelve human cancer cell lines, representing five epithelial cancer types (colon, pancreatic, liver, gastric, and cervical cancers), were selected for analyses. Tumor xenografts were grown in athymic nude mice and harvested once they reached approximately 0.4 cm³. To analyze the structure of the tumor vasculature, frozen sections were stained with anti-CD31 antibody, a marker specific for endothelial cells. Significant heterogeneity was observed in microvessel density (MVD) and endothelial tube length in the twelve different xenografts (Table 1).

TABLE 1
Microvessel density (MVD), endothelial tube length, and endothelial
tube coverage by pericytes, in 12 cancer xenografts.

| | | | Percent coverage by | Percent coverage by |
|---|---|---|---|---|
| Cell line | MVD (%) | Tube length (microns) | SMA expressing pericytes | NG2 expressing pericytes |
| CAPAN-1 | 7.33 ± 0.04 | 121.9 ± 34.1 | 49.0% | 72.7% |
| HCT116 | 3.21 ± 0.03 | 120.9 ± 21.6 | 29.8% | 92.7% |
| HEP3B | 4.13 ± 0.038 | 112.4 ± 48.4 | 35.4% | 47.9% |
| HT29 | 12.51 ± 0.07 | 55.0 ± 17.4 | 84.8% | 90.0% |
| KM12L4 | 4.61 ± 0.07 | 130.7 ± 28.1 | 45.3% | 72.0% |
| LOVO | 4.76 ± 0.03 | 100.9 ± 27.1 | 70.5% | 76.5% |
| LS174T | 4.48 ± 0.04 | 158.6 ± 36.1 | 53.2% | 97.7% |
| MKN45 | 2.40 ± 0.03 | 122.8 ± 34.5 | 53.4% | 75.8% |
| RKO | 1.10 ± 0.01 | 44.3 ± 19.8 | 90.5% | 69.8% |
| SIHA | 3.07 ± 0.01 | 80.1 ± 18.8 | 62.3% | 64.2% |
| SNU398 | 10.27 ± 0.14 | 267.0 ± 99.1 | 62.0% | 64.4% |
| SW480 | 2.10 ± 0.02 | 46.8 ± 11.4 | 28.6% | 39.3% |

Some tumors displayed high MVD and long endothelial tubes, while others displayed low MVD and short endothelial tubes. Pericytes are mural cells that are characterized by their distinctive shape and location surrounding endothelial tubes. Several lines of evidence have suggested that pericytes stabilize tumor endothelial cells against anti-VEGF therapy. However, when both endothelial cells and pericytes were targeted by inhibitors, there was loss of both immature and mature vessels (Bergers et al., 2003; Takagi et al., 2003).

The percentage of tumor vessels that were covered by pericytes was further determined. Tumor xenograft sections were double stained with anti-CD31 antibody for endothelial cells, and anti-NG2 or anti-SMA antibodies for pericytes and examined under fluorescence microscopy. It was found that endothelial coverage with SMA-expressing pericytes ranged from approximately 29.8-90.5%, and endothelial coverage with NG2-expressing pericytes ranged from approximately 39.3-97.7% (Table 1). In total, the data in Table 1 demonstrate that tumor xenografts display heterogeneity in their vasculature and interactions with pericytes.

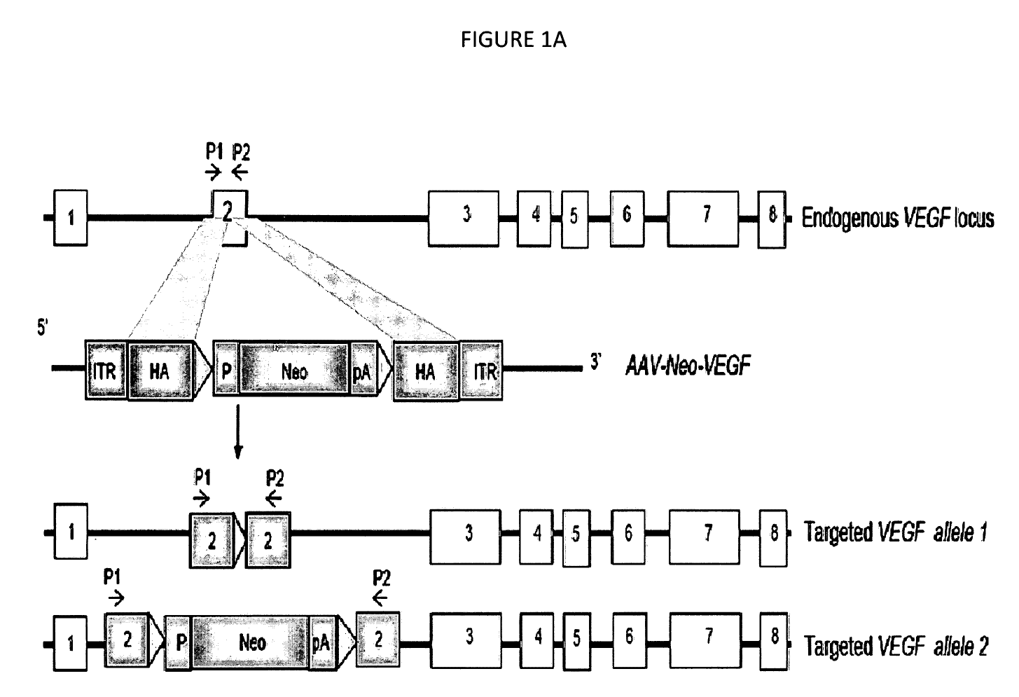

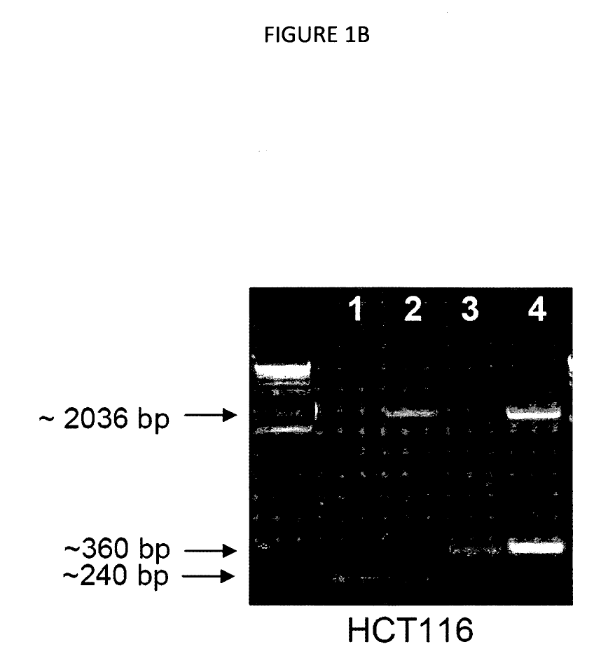

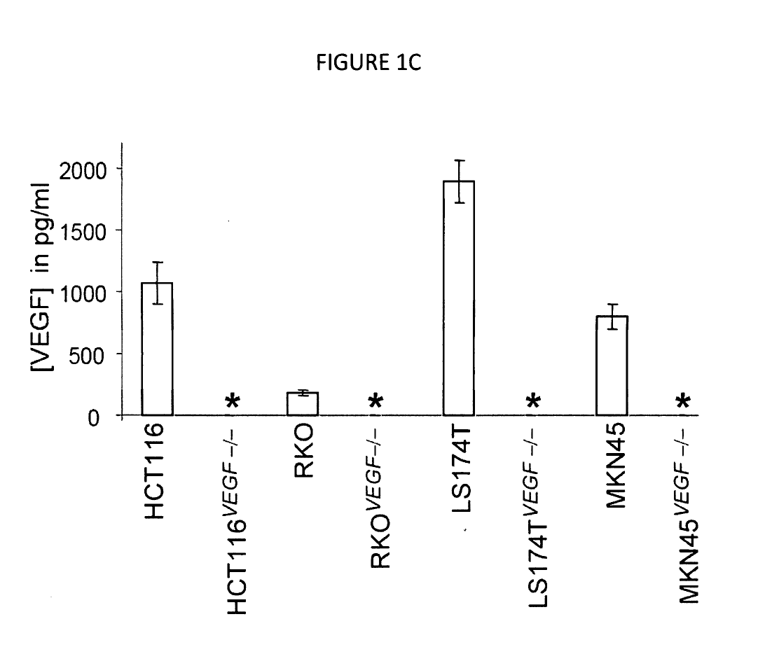

In developing embodiments of the present invention, it was contemplated that VEGF withdrawal leads to two types of tumor vessel response. To model effective antiangiogenic therapy, as well as control for potential differences in perfusion across various vessel types, the VEGF gene was disrupted by homologous recombination (FIG. 1). The human cancer cell lines HCT116, RKO, LS174T, and MKN45 were selected as they represent a wide variance in tumor vasculature (Table 1). Exon 2 of the VEGF gene loci was targeted for disruption (FIG. 1A). Since exon 2 is upstream of all potential VEGF alternative splicing sites, no VEGF products are predicted to be translated. Disrupted genotypes and loss of the VEGF gene product were confirmed by locus specific PCR and ELISA specific for VEGF protein, respectively (FIGS. 1B and 1C).

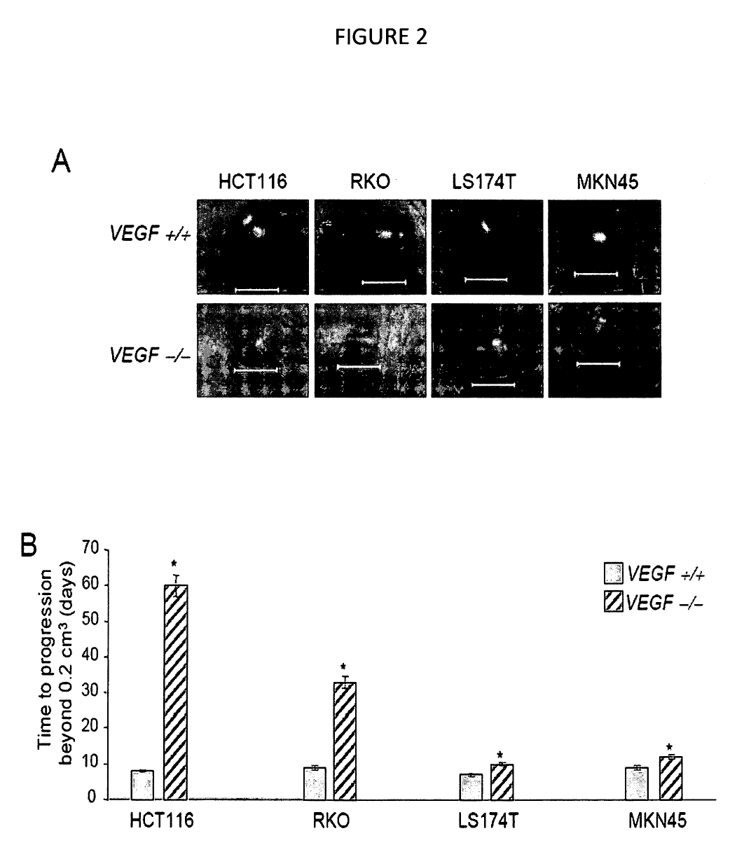

To determine the effects of VEGF disruption on tumor growth, cells were implanted into athymic nude mice to form xenografts. The overlying skin in the subcutaneous xenograft model showed blanching for the VEGF−/− xenografts, consistent with decreased vascular permeability (FIG. 2A). Examination of tumor volumes revealed that HCT116^{VEGF-/-} and RKO^{VEGF-/-} xenografts showed marked delay in tumor growth, in comparison to their respective parental controls (FIG. 2B). In contrast, smaller differences in xenograft growth delay were noted in the LS174T^{VEGF-/-} and MKN45^{VEGF-/-} xenografts, when compared with their respective parental controls (FIG. 2B). To determine whether the differences in tumor xenograft growth delay were secondary to differences in the effects of VEGF disruption on tumor MVD, tumor sections were stained with the endothelial cell marker CD31. Significant differences in changes in MVD were found in the various VEGF−/− xenografts compared to their respective parental controls. HCT116^{VEGF-/-} xenografts showed the most significant decreases in MVD compared to its parental control. RKO xenografts had the least MVD to begin with, which was then followed by very low residual MVD after disruption of VEGF. When endothelial tube length was measured, HCT116^{VEGF-/-} and RKO^{VEGF-/-} xenografts had only short, truncated residual vessels. LS174T^{VEGF-/-} and MKN45^{VEGF-/-} xenografts, on the other hand, had higher residual MVD, marked by both short and long residual vessels.

To determine if the residual vessels were functional, tumor perfusion was tested by intravenously injecting Hoechst 33342 into nude mice bearing parental versus VEGF−/− tumors. HCT116^{VEGF-/-} and RKO^{VEGF-/-} xenografts had significantly decreased perfusion. In fact, most of the perfusion was seen at the tumor capsule. In contrast, LS174T^{VEGF-/-} and MKN45^{VEGF-/-} xenografts maintained tumor perfusion, compared to their parental counterparts. These findings demonstrate that functional tumor vasculature was disrupted in HCT116^{VEGF-/-} and RKO^{VEGF-/-} xenografts, whereas it was preserved in LS174T^{VEGF-/-} and MKN45^{VEGF-/-} xenografts. Altogether, these findings demonstrate that some clinical tumors respond to antiangiogenic therapy by vessel normalization, and others by vessel regression.

In developing embodiments of the present invention, vascular determinants associated with the types of antiangiogenic response were investigated. Endothelial-pericyte (E-P) interactions were investigated for their affect on tumor vessel response in the various parental and VEGF−/− xenografts. Endothelial cells were stained with anti-CD31 antibody, and pericytes with anti-NG2 or anti-SMA antibodies. All VEGF−/− xenografts, when compared to their parental xenografts, had significantly decreased pericytes-free endothelial tubes, thereby demonstrating that disruption of VEGF effectively inhibited new endothelial sprouting amongst all the xenografts. Further, the abundance of SMA-staining pericytes was correlated with the abundance of CD31-staining vascular structures in the VEGF−/− xenografts. For example, LS174T^{VEGF-/-} and MKN45^{VEGF-/-} xenografts had more abundant SMA-staining pericytes and higher associated residual endothelial tubes than HCT116^{VEGF-/-} and RKO^{VEGF-/-} xenografts. These data confirm previous findings that SMA-staining pericytes protect endothelial cells. In addition, these data suggest that the relative quantity of SMA-staining pericytes in xenografts may determine the types of tumor vessel response. In contrast, although there were abundant NG2-staining pericytes in some xenografts, most were not associated with endothelial tubes (e.g., MKN45^{VEGF-/-} and HCT116^{VEGF-/-} xenografts). Furthermore, the relative abundance of NG2-staining pericytes did not correlate with the extent of tumor vessel regression. For example, while both MKN45^{VEGF-/-} and HCT116^{VEGF-/-} xenografts harbored abundant NG2-staining pericytes, they had distinct extents of tumor vessel regression.

As such, these data suggest that, among others, these two factors, basal tumor MVD and quantity of SMA-staining pericytes, predict how tumors would respond to antiangiogenic therapy. It is contemplated that these two measurements indicate the density of well-protected endothelial tubes in a tumor.

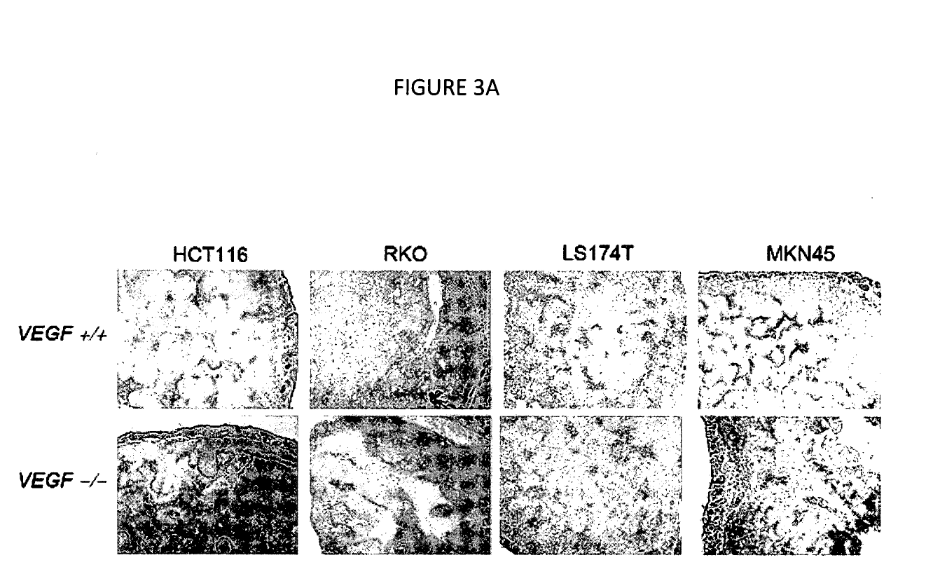

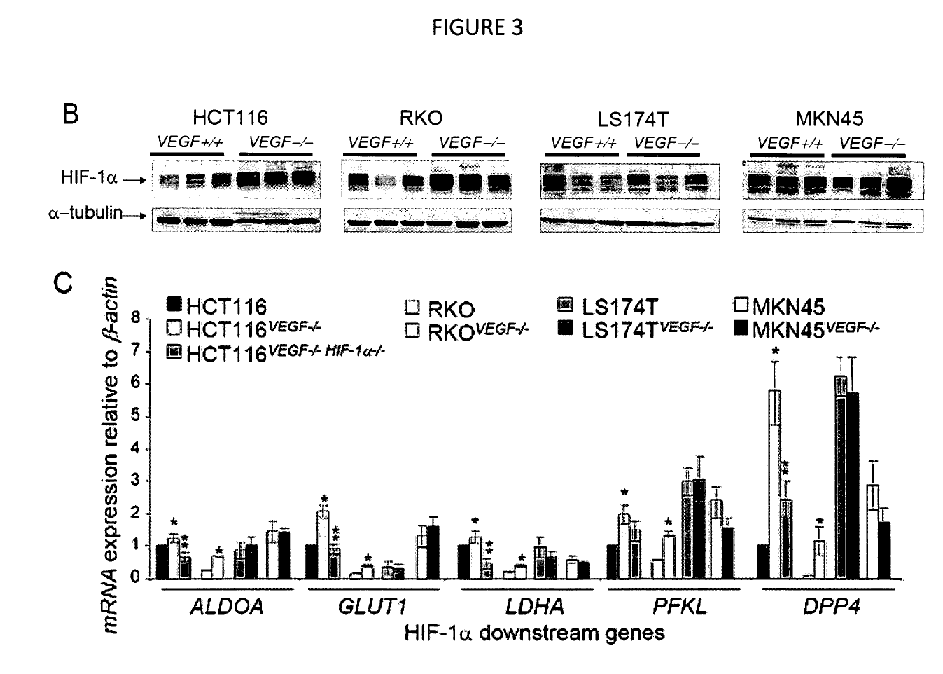

In developing embodiments of the present invention, the disruption of VEGF on increasing intratumoral hypoxia, the stabilization of HIF-1α, and induction of HIF-1 target genes in tumors with inadequate vessels was investigated. Experiments were performed to investigate whether the two different antiangiogenic responses were associated with changes in intratumoral hypoxia. Intratumoral hypoxia was determined by tumor uptake of the hypoxia marker pimonidazole (FIG. 3A). HCT116^{VEGF-/-} and RKO^{VEGF-/-} xenografts, which were characterized by residual short vessels and poor perfusion, and exhibited marked expansion of the tumor hypoxic compartments when compared to their respective parental xenografts. On the other hand, LS174T^{VEGF-/-} and MKN45^{VEGF-/-} xenografts, which were characterized by longer vessels and maintained perfusion, had no noticeable changes in intratumoral hypoxia, when compared to their respective parental xenografts.

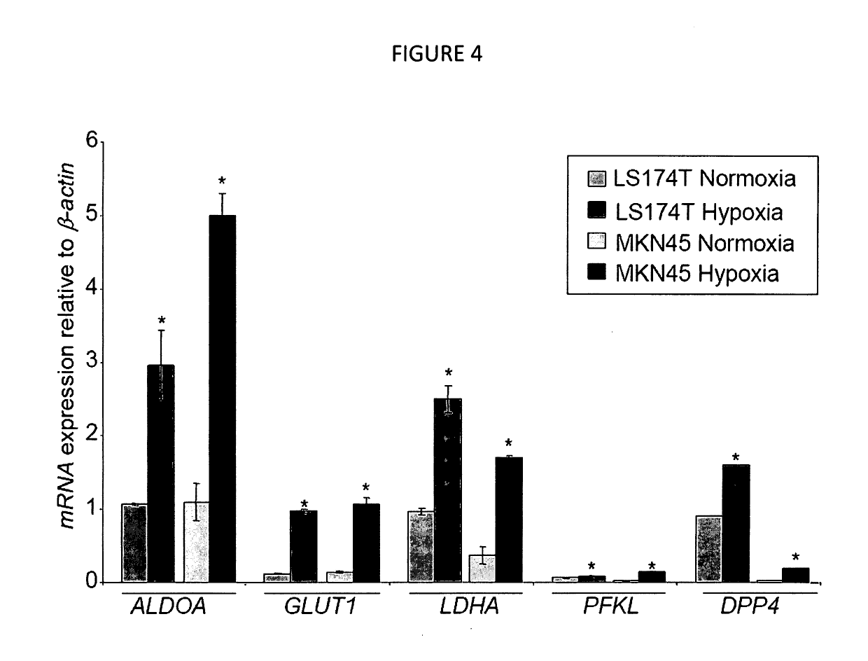

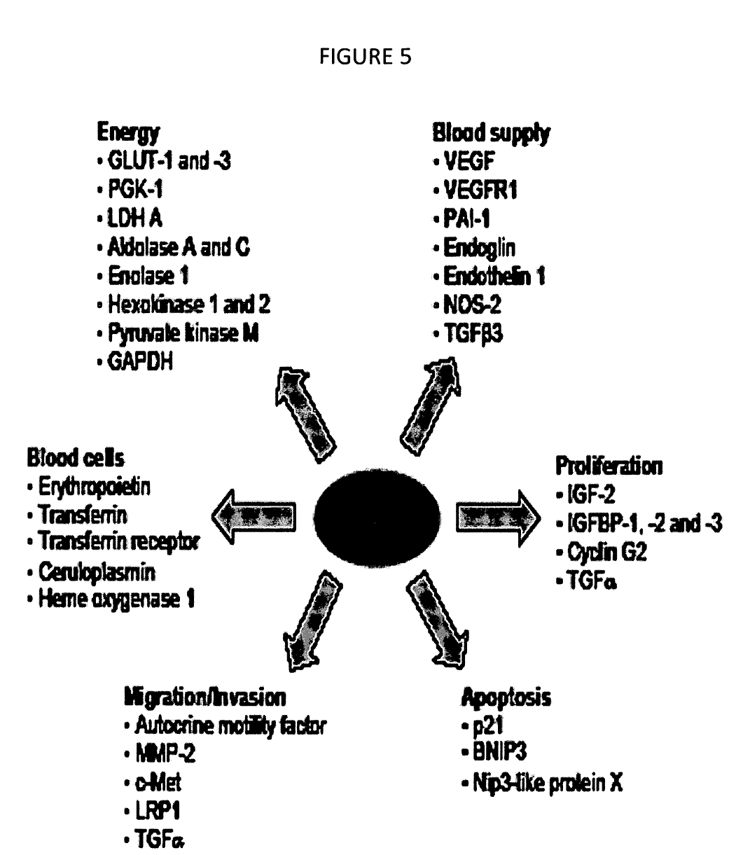

Tumor hypoxia leads to a reactive response in the tumor epithelial cells, wherein said cells typically become resistant to chemo and radiotherapy. This response is dominated by the induction of hypoxia-induced transcription factor HIF-1 target genes and HIF-1α stabilization. In normal cells, the balance between the synthesis and degradation of HIF-1α regulates the activity of HIF-1 under normoxic conditions. However, in many cancers the balance is deregulated because of the activation of the oncogenic pathways (e.g., phsophatidylinositol 3-kinase, MAP kinase) and the loss of tumor suppressor function (e.g., PTEN, p53) causing HIF-1 to accumulate regardless of oxygen concentration (Belozerov and Van Meir, 2006, Curr. Opin. Inv. Drugs 7:1067-76). HIF-1 is composed of the HIF-1α and HIF-1β subunits. Whereas HIF-1β is constitutively expressed, HIF-1α protein stability and synthesis are regulated by intratumoral hypoxia and genetic alterations. The HIF-1 complex transactivates over 70 target genes (FIG. 5), many of which are critical for tumor survival and progression, including, but not limited to, those important for two universal characteristics of solid tumors: angiogenesis and glycolysis. As such, HIF-1α expression in the parental and VEGF−/− xenografts was determined (FIG. 4B) using Western blot techniques. Compared to their parental xenografts, HCT116^{VEGF-/-} and RKO^{VEGF-/-} xenografts had increased expression of HIF-1α consistent with its stabilization under hypoxia upon VEGF inhibition. Conversely, there were no changes in HIF-1α expression in LS174T^{VEGF-/-} and MKN45^{VEGF-/-} xenografts, compared to their respective parental xenografts, consistent with the lack of oxygenation changes in these sets of xenografts upon VEGF inhibition.

The expression of four known HIF-1 target genes: ALDOA, GLUT1, LDHA, and PFKL were further examined. As shown in FIG. 3C, there was significant increase in the expression of all four HIF-1 target genes in HCT116^{VEGF-/-} and RKO^{VEGF-/-} xenografts, in comparison to the parental HCT116 and RKO xenografts. The induction of all four genes is HIF-1 dependent, as disruption of HIF-1α in the HCT116^{VEGF-/-} xenografts reversed the induction of these genes (HCT116^{VEGF-/-HIF-1α-/-} versus HCT116^{VEGF-/-} xenografts (FIG. 4C). In contrast, LS174T^{VEGF-/-} and MKN45^{VEGF-/-} xenografts, compared to their respective parental xenografts, had no statistically significant changes in the expression of all four HIF-1 target genes (FIG. 4C). The induction of GLUT1 was further tested by immunohistochemistry. For HCT116^{VEGF-/-} and RKO^{VEGF-/-} xenografts, in comparison to the respective parental xenografts, there was a significant increase in the relative proportion of intratumoral regions with GLUT1 expression, overlapping intratumoral hypoxic regions by analysis of serial sections. For LS174T^{VEGF-/-} and MKN45^{VEGF-/-} xenografts, in comparison to their respective parental xenografts, there was no statistically significant changes in the expression of all four HIF-1 target genes by real time PCR and no noticeable change in the expression of the GLUT1 protein. These data demonstrate that stabilization of HIF-1α, and the induction of HIF-1 target genes, are indicators of vessel regression and decreased tumor perfusion in response to antiangiogenic therapy. Conversely, the absence of HIF-1α stabilization, and the lack of induction of HIF-1 target genes, are indicators of maintenance of tumor blood vessels and continued tumor perfusion.

In developing embodiments of the present invention, experimentation was performed to identify novel HIF-1 target genes that are induced upon VEGF inhibition, wherein HCT116^{VEGF-/-} and HCT116^{VEGF-/-HIF-1α-/-} xenografts were subjected to global gene expression analyses using Affymetrix U133A GENECHIP (Santa Clara, Calif., USA). Genes were screened for at least 2-fold decreased expression in HCT116^{VEGF-/-HIF-1α-/-} xenografts, compared to HCT116^{VEGF-/-} xenografts. As HIF-1 target genes are induced by the binding of HIF-1 to hypoxia-response element (HRE) in their promoters, genes were screened for which contained an HRE sequence [(A/G)CGTG].

CD26, also known as DPP4, was identified as one of the down-regulated genes by loss of HIF-1α. CD26 is a 110-kDa glycoprotein that is expressed on numerous cell types, and characterized by its Dipeptidyl Peptidase IV (DPPIV or DPP4) enzymatic activity and plays an important role in T-cell stimulation. As such, CD26 has multiple biological functions, including glucose homeostasis, immune regulation, signal transduction, and apoptosis. CD26 has described roles in cancer progression and has been shown to be measurable in serum (see U.S. Pat. No. 7,198,788, herein incorporated by reference in its entirety). To validate the microarray results that CD26 is a putative HIF-1 target gene, and a potential biomarker for HIF-1 inhibition and antiangiogenic therapies and treatments, CD26 expression was evaluated by real-time RT-PCR in tumor lysates derived from our panel of parental and VEGF−/− xenografts, and also HCT116 VEGF-1 and HCT116^{VEGF-/-HIF-1α-/-} xenografts (FIG. 3C). In tumors in which HIF-1α is stabilized upon VEGF inhibition (HCT116 and RKO), there was significant induction of CD26, higher than other known HIF-1 target genes tested. The induction of CD26 was HIF-1 dependent, as there was suppression of expression in HCT116^{VEGF-/-HIF-1α-/-} xenografts, compared to HCT116^{VEGF-/-} xenografts. In contrast, in tumors without changes in HIF-1α expression upon VEGF inhibition, LS174T^{VEGF-/-} and MKN45^{VEGF-/-}, there was no induction of CD26 (FIG. 4C). Notably, LS174T and MKN45 cells are capable of HIF-1α stabilization, and CD26 induction, under in vitro hypoxic culture conditions (FIG. 4). Altogether, these data demonstrate that the induction of CD26 (DPP4) in tumors is an indicator of HIF-1α stabilization and down regulation of CD26 is an indicator of HIF-1 inhibition, and thus is useful as a marker of tumor vessel response, and the type of tumor vessel response, to antiangiogenic therapy.

Certain illustrative embodiments of the invention are described below. The present invention is not limited to these embodiments.

In some embodiments, the present invention provides methods (and associated systems, kits, and compositions for carrying out such method) utilizing CD26, HIF-1, or HIF-1 pathway members as a biomarker for antiangiogenic therapies. Antiangiogenic therapies comprise the administration of antiangiogenic drugs, compounds, small molecules, nucleic acids (e.g., RNAi constructs, etc.), and the like to treat angiogenic related cancers and diseases. Antiangiogenic drugs include, but are not limited to, Macugen (pegaptanib sodium), Lucentis (ranibizumab), Tryptophanyl-tRNA synthetase (TrpRS), Retaane (anecortave acetate), Combretastin A4 Prodrug (CA4P), AdPEDF, VEGF-TRAP, AG-013958, Avastin (bevacizumab), JSM6427, TG100801, ATG3, Sirolumus (rapamycin), OT-551 and Neovastat. Additional examples of antiangiogenic drugs and compounds can be found in the following patents, all of which are incorporated herein by reference; U.S. Pat. Nos. 5,972,896, 5,981,484, 6,376,525, 6,251,867, 6,248,327, 5,849,742, 6,371,905, 7,067,317, 7,026,462, 6,235,716, 7,176,289 and 7,135,192. In some embodiments, CD26, HIF-1, or HIF-1 pathway member expression or activity is measured in a sample, for example blood, serum, or plasma, in tissues, tumor tissues, tumor lysates, tissue biopsies, and the like.

In some embodiments, expression is determined in vitro in a sample using protein identification technologies such as Western Blot, fluorescence hybridization, and the like. In some embodiments, expression is determined in a sample by polymerase chain reaction or reverse-transcription polymerase chain reaction, although the present invention is not limited by the means of detection. In some embodiments, CD26 expression in a sample is increased over that of a control sample, thereby indicating, for example, HIF-1α stabilization. In some embodiments, the increased CD26 expression serves to identify an antiangiogenic treatment as one where, for example, normalization of abnormal tissue vasculature is occurring. In some embodiments, CD26 expression in a sample is decreased over that of a control sample, thereby indicating, for example, inhibition of HIF-1. In some embodiments, the decreased CD26 expression serves to identify an antiangiogenic treatment as one where, for example, vessel regression is occurring and oxygenation levels of the tissue are decreasing or have decreased causing hypoxic conditions. In some embodiments, therefore, CD26 is a biomarker for the determination of the different types of antiangiogenic response elicited by an antiangiogenic therapy regimen.

In some embodiments, the present invention provides methods and systems for monitoring progression of tumor antiangiogenic response, thereby, for example, furnishing a clinician with tools to select the most appropriate antiangiogenic agent(s) for a subject. For example, two classes of antiangiogenic agents are shown to be clinically efficacious. Bevacizumab, when added to standard chemotherapy in phase III clinical trials, led to increased response rate and prolonged progression-free survival. In contrast, monotherapy with bevacizumab has not shown significant survival benefit, in comparison to standard chemotherapy alone group. However, bevacizumab may have clinical benefit as monotherapy in a small minority of patients. It is possible that bevacizumab predominantly acts through “vessel normalization”, and improves the efficacy of chemotherapy; and only occasionally causes vessel disruption. In contrast, multitargeted tyrosine kinase inhibitors (TKIs) have not improved efficacy when combined with chemotherapy. However, multitargeted TKIs, such as sunitinib and sorafenib, showed clinical benefit as monotherapy. Since multi-targeted TKIs target PDGFR-β and inhibit pericytes, these agents might create inadequate, poorly perfusing vessels, and thereby cause tumor regression. Currently, there are no proven biomarkers to predict or monitor the efficacy of antiangiogenic therapy in patients. For example, tumors that are predicted to develop a “vessel normalization” type of antiangiogenic response (i.e. LS174T and MKN45), would benefit from combination therapies with bevacizumab plus standard chemotherapy. When these tumors progress, TKIs can then be used to induce vessel regression. In contrast, tumors that are predicted to develop a vessel regression type of antiangiogenic response (i.e. RKO and HCT116), would benefit from therapy with bevacizumab or TKIs alone. Monitoring CD26 levels as a biomarker for the antiangiogenic response, as embodied in the methods and systems of the present invention, determines the antiangiogenic type of response in a subject when confronted with mono or multichemotherapeutic regimens, thereby helping clinicians decide on a plan of attack in treating a subject.

In some embodiments, the biomarkers of the present invention are used to monitor antiangiogenic therapies that are ongoing or just beginning. In some embodiments, the monitoring of the progress of a drug or treatment regimen using methods and systems of the present invention provides for a clinician to render a prognosis based on the efficacy of the treatment regimen. In some embodiments, the prognosis prompts a clinician to augment a particular subject's treatment regimen to a treatment regimen that is more efficacious in dealing a particular subject's tumor, wherein the progress of the antiangiogenesis efficacy of the new treatment regimen is again monitored by the methods and systems of the present invention.

In some embodiments, the present invention provides for the monitoring of HIF-1 activity in tumors. In some embodiments, methods and systems of the present invention comprise the monitoring of HIF-1 activity in tissues by monitoring CD26 expression in the tissues. In some embodiments, CD26 expression is monitored as an indicator of HIF-1 target gene induction caused by, for example, hypoxia. CD26 expression, for example, increases upon HIF-1 induction, as CD26 is a HIF-1 target gene as herein demonstrated. As such, in some embodiments, HIF-1 induction is characterized by the CD26 biomarker wherein increase in expression of CD26 in a sample is indicative of hypoxia in a tumor. In some embodiments, CD26 is used as a biomarker for HIF-1 induction. Conversely, in some embodiments, CD26 is useful in monitoring anti-HIF-1 therapy, or HIF-1 inhibition by clinical therapies (e.g., drugs, small molecules, compounds, nucleic acids, etc.), either antiangiogenic or otherwise.

In some embodiments, the present invention provides for the monitoring of HIF-1 targeted therapies by CD26 expression. HIF-1 targeted therapies include, but are not limited to, inhibitors of HIF-1. Inhibitors of HIF-1 include, but are not limited to, those found in U.S. Pat. Nos. 7,205,283 and 6,753,321, 2005/0119243 and gefitinib (Pore et al., 2006, Cancer Res. 66:3197-204), LY-294002 (Jiang et al., 2001, Cell Growth Diff. 12:363-9), Wortmannin (Jiang et al., 2001), rapamycin (Hudson et al., 2002, Mol. Cell. Biol. 22:7004-14), CCl-779 (Wan et al., 2006, Neoplasia 8:394-401), Rad-001 (Majumder et al., 2004, Nat. Med. 10:594-601), topotecan (Rapisarda et al., 2004, Cell Cycle 3:172-5), 103D5R (Tan et al., 2005, Cancer Res. 65:605-12), geldanamycin (Mabjeesh et al., 2002, Cancer Res. 62:2478-82), 17-AAG (Ibrahim et al., 2005, Cancer Res. 65:11094-100), 2-methylestradiol (Mabjeesh et al., 2003, Cancer Res. 3:363-75), PX-12 (Welsh et al., 2003, Mol. Cancer. Ther. 2:235-43), pleurotin (Welsh et al., 2003), PX-378 (Welsh et al., 2004, Mol. Cancer. Ther. 3:233-44), chetomin (Kung et al., 2004, Cancer Cell 6:33-43), NSC-50352 (Park et al., 2006, Cell Cycle 5:1847-53), polyamides (Olenyuk et al., 2004, Proc. Natl. Acad. Sci. 101:16768-773; Viger et al., 2006, Bioorg. Med. Chem. 14:8539-49) and echinomycin (Kong et al., 2005, Cancer Res. 65:9047-55). Anti-HIF-1 therapies are frequently combined with known chemo and radiation therapies for cancers and diseases. HIF-1 related diseases for which HIF-1 related therapies are important include, but are not limited to, cancers, cardiovascular remodeling, preeclampsia, aging and aging related diseases, arthritis, and ischemic disorders (Park et al., 2004, J. Pharm. Sci. 94:221-32). As such, in some embodiments the methods and systems as described herein provide for determining the efficacy of inhibition of HIF-1 by monitoring the presence or absence of CD26 in tumor tissues. However, the present invention is not limited to CD26. For example, any HIF-1 target gene that is measurable and correlated in its response to HIF-1 inhibition is a suitable biomarker for methods and systems of the present invention. In some embodiments, the present invention provides diagnostics for HIF-1 associated disease detection.

In some embodiments, methods of the present invention provide for identifying test compounds and therapeutics useful as antiangiogenic therapies and treatments for cancer and angiogenic related diseases. In some embodiments, a test compound monitored using the CD26 biomarker wherein administration of said test compound either increases the expression of CD26 (e.g., normalization of abnormal tumor vessels) or decreases the expression of CD26 (e.g., vessel regression and/or hypoxia) in a subject.

In some embodiments, the present invention provides systems (e.g., kits) for assaying for biomarker expression in a sample for use in monitoring antiangiogenic therapies and treatments. In some embodiments, a system for assaying biomarker expression comprises reagents for performing in vitro protein based assays on tissue biopsies or tissue lysates including, but not limited to probes either fluorescently labeled or not, reagents, buffers and the like. Protein based in vitro assays include, but are not limited to Western Blots, Enzyme-linked Immunosorbent Assays, in situ tissue assays, luminescent and/or calorimetric enzymatic assays, and the like. In some embodiments, a system for assaying for biomarker expression in monitoring antiangiogenic therapies and treatments comprises reagents for performing in vitro nucleic acid based assays. In such embodiments, primers, reagents, buffers and the like useful, necessary, or sufficient to perform, for example, polymerase chain reaction or reverse-transcription polymerase chain reaction on biomarker DNA or RNA are provided.

### EXPERIMENTATION

The following examples are provided in order to demonstrate and further illustrate certain preferred embodiments and aspects of the present invention and are not to be construed as limiting the scope thereof.

Cell lines utilized in embodiments of the present invention include HCT116, RKO, and LS174T human colon cancer cell lines acquired from the American Type Culture Collection (Manassas, Va.). MKN45 human gastric cancer cell line was acquired from the Japanese Collection of Research Bioresources/Human Science Research Resources Bank (Osaka, Japan). Cells were cultured in McCoy5A media, supplemented with 10% FBS and 1% penicillin/streptomycin (Invitrogen).

### Example 1

### In Vivo Tumorigenesis and Assays

Parental and VEGF^{-/-} cells were grown in complete media and harvested for in vivo studies as previously described (Dang et al., 2001; Dang et al., 2004). Six-week old female athymic nu/nu mice (Charles River Labs, Wilmington, Mass.) were implanted subcutaneously into the flanks with approximately 7.5×10⁶ cells, as previously described (Dang et al., 2006, Cancer Res. 66:1684-936). Tumor sizes in two dimensions were measured with calipers, and volumes were calculated with the formula (L×W²)×0.5, where L is length and W is width. Student's paired t-test was used to determine statistical significance between groups. Mice were housed in barrier environments, with food and water provided ad libitum. Xenografts were harvested for subsequent analyses when they reached approximately 0.4 cm³.

Harvested xenografts were fixed in Tissue-Tek OTC compound (Sakura Finetek, Torrance, Calif.) and stored at −80° C. Frozen sections, 10 microns in thickness, were prepared with a Leica Microsystems cryostat. For tumor microvessel density determination, sections were sequentially incubated with a monoclonal antibody against endothelial cells-specific marker CD31 (Pharmingen), followed by a biotinylated secondary antibody (Jackson ImmunoResearch Laboratories), and rhodamine-streptavidin (Vector). For double and triple immunofluorescence staining, sections were incubated with primary antibodies which were raised in different species: CD31 (rat), NG2 (rabbit), or SMA (mouse). After washes, species-specific secondary antibodies coupled to Rhodamine or FITC were applied. The absence of crossreactivity amongst secondary antibodies against primary antibodies was verified by omitting one primary antibody during the first incubation. At least three sections of each of three tumors, representing middle and distal parts of the tumor, were examined under fluorescence microscopy, and representative results are illustrated in the figures.

Images were captured on SPOT software and analyzed with Image-Pro Plus software. To determine microvessel density (MVD) the percent of the image field that stained with specific antibodies was measured. To determine endothelial tube length, at least 20 tubes were traced and measured. To determine endothelial coverage by pericytes, images were superimposed, and the percent of overlapping or adjacent staining were measured. All measurements were made in at least 9 separate images and averaged.

### Example 2

### Disruption of the Human VEGF and HIF-1α Gene

The endogenous locus, adeno-associated virus (AAV) knockout construct, and resulting targeted locus are shown in FIG. 1A. The strategy is as previously described (Chan et al., 2002, Proc. Natl. Acad. Sci. 99:8265-70; Cummins et al., 2004, Cancer Res. 64:3006-8; Kohli et al., 2003, Nucl. Acids Res. 32:3-10). Exon 2 of VEGF was targeted for disruption with an AAV cassette containing the Neo resistance gene under the constitutive control of a SV40 promoter flanked by left and right homology arms approximately 1 kb in length. Cells exhibiting neomycin resistance were screened with locus-specific PCR to confirm homologous integration of the targeting vector. Once the first allele was successfully targeted, the Neo resistance gene was excised using Cre recombinase. The same targeting vector was used to target the second allele. For locus-specific PCR, genomic DNA was amplified using primers specific for exon 2. Loss of VEGF was confirmed by ELISA, wherein an equal numbers of cells were plated overnight. The VEGF protein level in cultured medium was analyzed using the Quantikine VEGF ELISA Kit (R & D Systems, Minneapolis, Minn.) following manufacturer's protocol. Disruption of HIF-1α is as previously described (Dang et al., 2006).

### Example 3

### Tumor Assessments

To access tumor vessel perfusion, mice bearing parental or VEGF−/− xenografts were intravenously injected with Hoescht 33342 (40 mg/kg), two minutes prior to sacrifice. Tumors were fixed in Tissue-Tek OTC compound (Sakura Finetek, Torrance, Calif.) and stored at −80° C. Frozen sections 10 microns in thickness were prepared with a Leica Microsystems cryostat and then examined under fluorescence microscopy.

To examine intratumor hypoxia, mice were administered the hypoxia marker pimonidazole, 60 mg/kg intraperitoneally 2 hours before sacrifice. Pimonidazole binds to the thiol-containing proteins specifically in hypoxic cells (Rofstad et al., 1999, Int. J. Radiat. Biol. 75:1377-93). Intraperitoneal injection of pimonidazole results in its uptake by hypoxic tumor cells; and bound pimonidazole can be detected in xenografts using antibody to pimonidazole.

For performing immunohistochemistry on xenograft tissues, harvested xenografts were fixed in formalin, paraffin embedded, sectioned, and stained with Hematoxylin and Eosin (H & E) by the University of Michigan Tissue Core Facility. Paraffin sections were deparaffinized, incubated with Proteinase K (Invitrogen), heated to 95oC for 20 min. in citrate buffer (pH6) and treated with peroxidase blocking reagent (Dako). In some studies, sections were incubated with a monoclonal antibody against glucose transporter-1 (glut-1, Dako) followed by a HRP-conjugated secondary antibody (Jackson ImmunoResearch Laboratories) and developed with diaminobenzidine (Sigma) staining. Bound pimonidazole was detected using the Hypoxyprobe-1 Plus kit (Chemicon International, Inc., Temecula, Calif.).

### Example 4

### Gene and Protein Expression Assessments

Gene expression profiling was performed wherein HCT116^{VEGF-/-} and HCT116^{VEGF-/-HIF-1α-/-} xenografts were harvested at approximately 0.4 cm³ and total RNA extracted. Gene expression analyses on the samples were performed at the University of Michigan Comprehensive Cancer Center Affymetrix Core Facility. Commercial high-density oligonucleotide arrays (GENECHIP Human Genome U133A; Affymetrix, Inc.) were used, following protocols and methods developed by the supplier. The human genome U133A chip consists of 22,283 probe sets of 25-base long single-stranded DNA sequences, each representing a transcript. Single stranded cDNA were synthesized and converted into double stranded cDNA. An in vitro transcription (IVT) reaction was carried out in the presence of biotinylated UTP and CTP to produce biotin-labeled cRNA. cRNA were then fragmented and hybridized to the test array, washed and stained with streptavidin-phycoerythrin, then scanned using the GeneArray scanner. Images were analyzed following quality control parameters provided by Affymetrix. The samples were hybridized to the standard array for 16 hours at 45° C., washed and stained using the fluidics station, then scanned. The images were analyzed using Microarray Suite Software and comparison analyses were carried out according to the instructions provided by Affymetrix. The first step in data analysis was to assure overall quality of the raw data. For each sample, the distribution of the perfect match (PM) probes were graphed (log intensity as a function of density), and RNA degradation plot was generated (Probe Number as a function of Mean Intensity:shifted and scaled. The shapes and slopes of all curves appeared similar, so the raw data were of high quality. The expression values for all the genes on each chip were computed using a robust multi-array average (RMA) and genes that appear not to be expressed were filtered. Comparisons were made using a 2.5-fold cutoff.

Real-time reverse transcription analysis was performed using extracted total RNA from cell lines or xenografts, treating with DNAse I (Qiagen, Valencia Calif.). Single stranded cDNA was generated using the iScript 1^{st} Strand Kit (BioRad, Hercules Calif. Real time PCR reactions were performed in triplicate on RT-derived cDNA, and relative values calculated as previously described (Pfaffl, 2001, Nucl. Acids Res. 29:2003-7). PCR products met three criteria to be included in the study; 1) the signal from the RT derived cDNA was at least 100 fold greater than that of control reactions performed without RT, 2) PCR products from the reactions with RT had to be the expected size on gel electrophoresis, and 3) melt curves analysis were consistent with specificity of PCR. Relative gene expression of aldolase, CD26, Glut1, LDH and PFK to β-actin were calculated using the formula as found in Pfaffl, 2001.

Western blot analysis was performed using whole-cell protein extracts, separated by electrophoresis, transferred to nitrocellulose membranes, and probed with antibodies as described previously (Dang et al., 2006). Antibodies were obtained from BD Transduction laboratories (San Jose, Calif.; mouse anti-human HIF-1α), Sigma (St. Louis, Mo.; α-tubulin), and Jackson Immunoresearch Laboratories (West Grove, Pa.; anti-mouse horseradish peroxidase). Antibody dilutions were as recommended by the manufacturer.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of determining a response to antiangiogenic treatment comprising:
a) assaying a sample from a subject undergoing antiangiogenic treatment for the expression of CD26, and
b) determining a response to antiangiogenic treatment based on the expression of CD26 in said sample relative to that of a control.

2. The method of claim 1, wherein said sample is a serum or a tissue sample.

3. The method of claim 2, wherein said tissue sample is a biopsy or a lysate.

4. The method of claim 1, wherein said subject is a mammal.

5. The method of claim 4, wherein said mammal is a human.

6. The method of claim 1, wherein said assaying comprises a protein assay.

7. The method of claim 1, wherein said assaying comprises a nucleic acid assay.

8. The method of claim 1, wherein said response to antiangiogenic treatment comprises normalization of abnormal tumor vessels.

9. The method of claim 8, wherein said normalization of abnormal tumor vessels comprises an increase in expression of CD26.

10. The method of claim 1, wherein said response to antiangiogenic treatment comprises tumor vessel regression.

11. The method of claim 10, wherein said tumor vessel regression comprises a decrease in expression of CD26.

12. A method of determining the efficacy of an antiangiogenic treatment comprising:
a) assaying a sample from a patient undergoing antiangiogenic treatment for the expression of CD26, and
b) determining the efficacy said antiangiogenic treatment based on the expression of CD26 in said sample relative to that of a control.

13. The method of claim 12, wherein an increase or decrease of expression of CD26 in said sample is indicative of increased or decreased efficacy of said antiangiogenic treatment.

14. A method for determining inhibition of HIF-1 by a compound comprising:
a) assaying a sample from a subject contacted with said compound for the expression of CD26, and
b) determining inhibition of HIF-1 based on the presence or absence of expression of CD26 in said sample as compared to that of a control.
